(19) United States
(12) Patent Application Publication
(10) Pub. No.: US 2020/0011003 A1
(43) Pub. Date: Jan. 9, 2020

(54) CLOSET APPAREL DRYING, DEHUMIDIFYING AND SANITIZING UNIT

(71) Applicant: ProCom Heating, Inc., Brea, CA (US)

(72) Inventor: David Deng, Diamond Bar, CA (US)

(73) Assignee: ProCom Heating, Inc., Brea, CA (US)

(21) Appl. No.: 16/030,537

(22) Filed: Jul. 9, 2018

(51) Int. Cl. D06F 58/10; A61L 2/10

(52) U.S. Cl. CPC D06F 58/10; A61L 2202/26; A61L 2/10

(57) ABSTRACT

A closet has the ability to dehumidify, heat and/or circulate the air therein in order to dry clothes. The dehumidification and circulation functions may be (1) included in a unitary closet structure, usable indoor or outdoor, (2) included as a modular unit for retro-fitting into an existing closet storage solution, or (3) a chassis that may be scaled and built into closets during their design and manufacture. The closet can include a sanitizing unit and may move air through a condenser, warm the dehumidified air, and deliver that treated air to a different portion of the closet, creating an air flow about the clothes hanging therein. A condensate collection unit may automatically shut down the system when a condensate reservoir is filled, thereby preventing condensate overflow. The closet may operate without the heat and/or dehumidification function, where external air may be circulated in and out of the closet.

### BACKGROUND OF THE INVENTION

### 1. Field of the Invention

Embodiments of the invention relates generally to clothes drying accessories. More particularly, the invention relates to a closet specifically configured for dehumidification, heating and/or circulation of air to effective dry clothes stored therein.

### 2. Description of Prior Art and Related Information

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

In situations where clothes need to hand to dry after washing, a person often has to hang the garments in open air first, to permit them to dry, then transfer to a closet. If the wet clothes are hung directly in the closet, moisture may build up in the closet, delaying drying of the clothes and creating the potential for other issues, such as mold growth in the closet. Clothes drying in open air may take a considerable amount of time, especially in humid conditions. Finally, clothes drying in open air, such as in an interior room in a home, can contribute to high humidity conditions within the home, requiring excess air conditioning, for example.

In view of the foregoing, there is a need for closet in which damp clothes may be disposed that avoids the above issues in conventional solutions.

### SUMMARY OF THE INVENTION

Embodiments of the present invention provide an apparel drying system comprising an interior space configured for receiving apparel therein; a dehumidifying unit having at least one condensing element; an air intake positioned at a first location in the interior space; a fan moving air from the interior space, through the air intake and over the at least one condensing element; and an air discharge grill receiving dehumidified air after passing through the dehumidifying unit, the air discharge grill positioned at a second location, within the interior space.

Embodiments of the present invention further provide an apparel drying closet comprising an interior space configured for receiving apparel therein; one or more doors for accessing the interior space; a dehumidifying unit having at least one condensing element; an air intake positioned at a first location in the interior space; a fan moving air from the interior space, through the air intake and over the at least one condensing element; an air discharge grill receiving dehumidified air after passing through the dehumidifying unit, the air discharge grill positioned at a second location, within the interior space; a condensate collection unit collecting condensate from the at least one condensing element; and a sanitizing unit emitting an ultraviolet light within the interior space.

Embodiments of the present invention provide an apparel drying system comprising an interior space configured for receiving apparel therein; an air intake positioned at a first location in the interior space; an air discharge grill positioned at a second location within the interior space; a fan moving air from the interior space, through the air intake and out of the air discharge grill; and cabinet air doors openable to permit external air to exchange with air inside the interior space, the cabinet air doors closable to exclude external air from inside the interior space.

Embodiments of the present invention also provide a method for drying apparel disposed within a closet comprising moving air from an interior space of the closet through an air intake and into a dehumidifying unit, the interior space configured to receive the apparel therein; condensing water out of the air via one or more condensing elements; and discharging dehumidified air back into the interior space through an air discharge grill.

These and other features, aspects and advantages of the present invention will become better understood with reference to the following drawings, description and claims.

### BRIEF DESCRIPTION OF THE DRAWINGS

Some embodiments of the present invention are illustrated as an example and are not limited by the figures of the accompanying drawings, in which like references may indicate similar elements.

FIG. 1 illustrates a partially cut-away perspective view of a closet having the apparel drying and sanitizing unit according to an exemplary embodiment of the present invention;

FIG. 2 illustrates a front view of the closet of FIG. 1;

FIG. 3 illustrates a cross-sectional view taken along line of FIG. 3;

FIG. 4 illustrates a perspective view of a modular, retrofit-type closet apparel drying and sanitizing unit according to an exemplary embodiment of the present invention;

FIG. 5 illustrates a front view of the modular unit of FIG. 4;

FIG. 6 illustrates a cross-sectional view taken along line VI-VI of FIG. 5;

FIG. 7 illustrates a rear perspective view of the dehumidifier module and control module of the modular unit of FIG. 4;

FIG. 8 illustrates a back view of the dehumidifier module and control module of FIG. 7;

FIG. 9 illustrates a front view of the dehumidifier module and control module of FIG. 7;

FIG. 10 illustrates a cross-sectional view taken along line X-X of FIG. 9;

FIG. 11 illustrates a stand-along closet having an apparel drying and sanitizing unit according to an exemplary embodiment of the present invention;

FIG. 12 illustrates a front view of a condensate reservoir, in a non-filled state, of an apparel drying and sanitizing unit according to an exemplary embodiment of the present invention;

FIG. 13 illustrates a cross-sectional view taken along line XIII-XIII of FIG. 12;

FIG. 14 illustrates a front view of a condensate reservoir, in a filled state, of an apparel drying and sanitizing unit according to an exemplary embodiment of the present invention;

FIG. 15 illustrates a cross-sectional view taken along line XV-XV of FIG. 14;

FIG. 16 illustrates a perspective view of a dryer/sanitizer apparel cabinet according to an exemplary embodiment of the present invention;

FIG. 17 illustrates a front view of the apparel cabinet of FIG. 16;

FIG. 18 illustrates a cross-sectional view taken along line XVIII-XVIII of FIG. 17;

FIG. 19 illustrates a partially cut-away perspective view of the apparel cabinet of FIG. 16;

FIG. 20 illustrates a cross-sectional view of the apparel cabinet of FIG. 16, showing an internal heat and dehumidification operation mode thereof;

FIG. 21 illustrates a cross-sectional view of the apparel cabinet of FIG. 16, showing an internal heat and external air exchange operation mode thereof; and

FIG. 22 illustrates a cross-sectional view of the apparel cabinet of FIG. 16, showing an external air exchange operation mode thereof.

Unless otherwise indicated illustrations in the figures are not necessarily drawn to scale.

The invention and its various embodiments can now be better understood by turning to the following detailed description wherein illustrated embodiments are described. It is to be expressly understood that the illustrated embodiments are set forth as examples and not by way of limitations on the invention as ultimately defined in the claims.

### DETAILED DESCRIPTION OF THE PREFERRED EMBODIMENTS AND BEST MODE OF INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention, and is not intended to limit the invention to the specific embodiments illustrated by the figures or description below.

As is well known to those skilled in the art, many careful considerations and compromises typically must be made when designing for the optimal configuration of a commercial implementation of any system, and in particular, the embodiments of the present invention. A commercial implementation in accordance with the spirit and teachings of the present invention may be configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

Broadly, embodiments of the present invention provide a closet having the ability to dehumidify, heat and/or circulate the air therein in order to dry clothes. The dehumidification and circulation functions may be (1) included in a unitary closet structure, usable indoor or outdoor, (2) included as a modular unit for retro-fitting into an existing closet storage solution, or (3) a chassis that may be scaled and built into closets during their design and manufacture. The closet apparel dryer with sanitizing unit may move air through a condenser, re-warm the dehumidified air, optionally further warming the air, and deliver that treated air to a different portion of the closet, creating an air flow about the clothes hanging therein. A condensate collection unit may automatically shut down the system when a condensate reservoir is filled, thereby preventing condensate overflow. The closet may operate without the heat or dehumidification function, where external air may be circulated in and out of the closet.

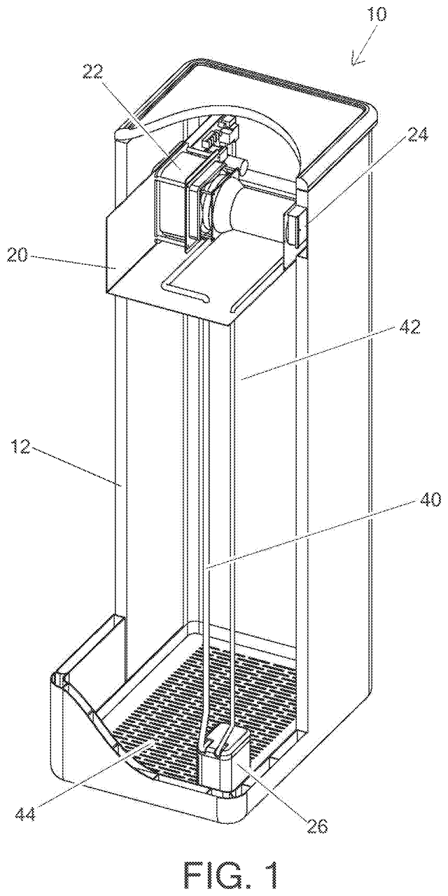

Referring now to FIGS. 1 through 3, a closet 10 can include a front side having doors 12 that open to an interior region 14. Clothes (not shown) may be disposed in the interior region 14, on hangers 16 disposed on a hanger rod 18, for example.

The closet 10 can include an apparel drying unit 20 that is operable to dehumidify and circulate air throughout the interior region 14. The apparel drying unit 20 can include a dehumidifier unit 22, heat transfer element 24 and condensate collection unit 26. The dehumidifier unit 22 can include one or more condensing elements 28 that is cooled to condense water from air, thereby dehumidifying the air. The condensing elements 28 may be cooled by conventional processes as may be understood by one skilled in the art.

The heat transfer element 24 may warm the air cooled after passing through the dehumidifier unit 22. In some embodiments, the heat transfer element 24 may warm the air to ambient air temperatures. In other embodiments, the heat transfer element 24 may warm the air warmer than ambient temperatures to aid in clothes drying. A control panel display 30 may include an adjuster for adjusting the temperature of the air warmed by the heat transfer element 24.

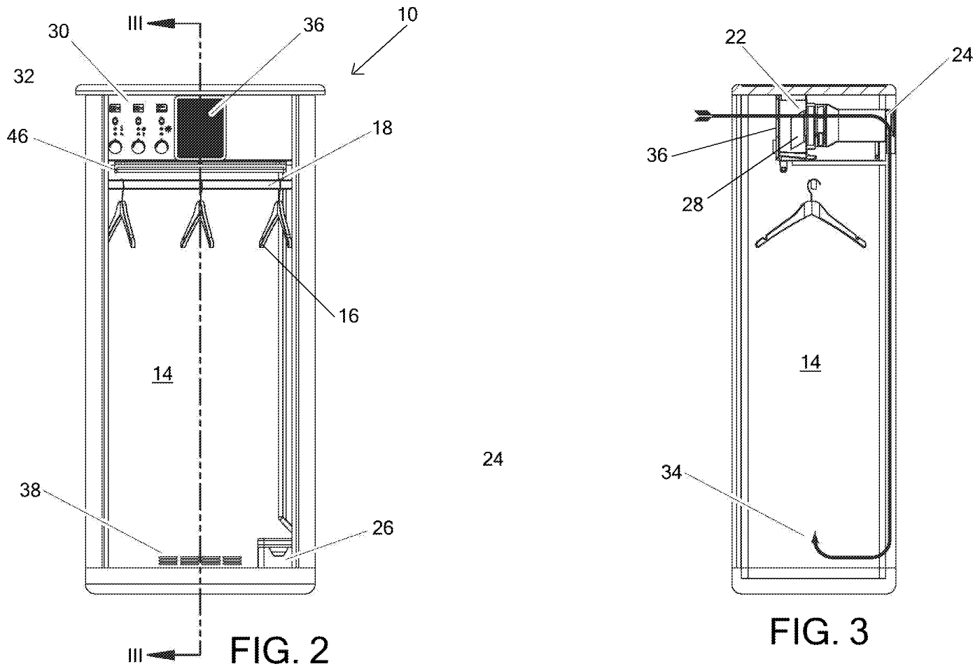

FIG. 3 shows an exemplary air flow pattern 34, passing through air intake filter 36, through the dehumidifier unit 22, the heat transfer element 24 and out at the bottom of the closet 10 through the air discharge grill 38 (see FIG. 2). Of course, other air flow patterns are contemplated within the scope of the present invention. For example, air discharge grills 38 may be disposed not only at the bottom of the closet 10, but also at one or more locations between the top and the bottom of the closet, for example. In some embodiments, the orientation of the air flow may be altered or even reversed, where the intake occurs at the bottom of the closet and the air outlets are disposed at an upper side of the closet. An air fan (not shown, see FIG. 8) may be used to move air through the apparel drying unit 20. The air fan may be disposed in the dehumidifier unit 22, pushing air through the system, or may be disposed near the air discharge grill 38, pulling air through the system.

The condensate collection unit 26 can collect liquid condensate released from the condensing elements 28. Typically, the condensate collection unit 26 is disposed at the bottom of the closet interior 14, but other locations are contemplated within the scope of the present invention. A condensate drain line 40 may carry the condensate into the condensate collection unit 26. A wiring harness 42 may run between the condensate collection unit 26 to the dehumidifier unit 22, where, when the condensate collection unit 26 is filled, as discussed below, a signal carried on the wiring harness 42 may shut down the system, thereby preventing overflow of the condensate collection unit 26.

In some embodiments, where the closet is a stand-along, unitized closet cabinet, or where the closet is custom built for a user, a drip tray catch basin 44 may be disposed on the bottom of the closet 10 in order to collect water that may drip from clothes hung in the closet 10.

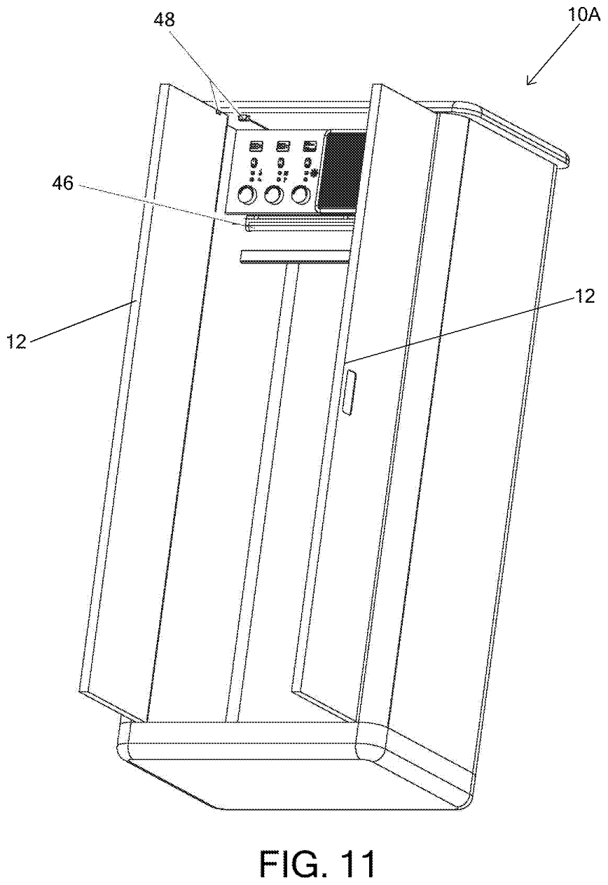

A sanitizer 46 may be optionally disposed in the closet. The sanitizer 46 can include a UV lamp that can illuminate inside the closet 10 to sanitize clothing. While the Figures show the sanitizer 46 at the top of the closet, various placement configurations for UV lamps are contemplated within the scope of the present invention. Referring also to FIG. 11, a movable unitary closet 10A can include the features discussed above. In FIG. 11, there is shown a door proximity safety switch 48. In some embodiments, there may be a switch 48 for each door 12. The switch 48 may turn off the sanitizer 46 when either of the doors 12 are opened, thereby preventing the user from contacting UV light generated by the sanitizer

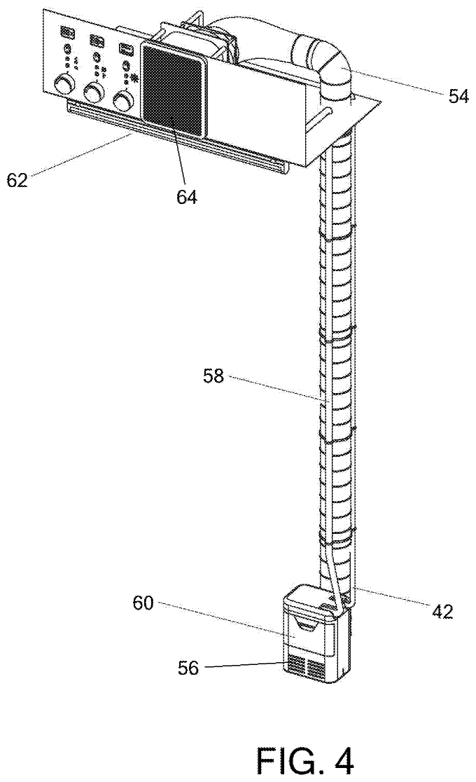

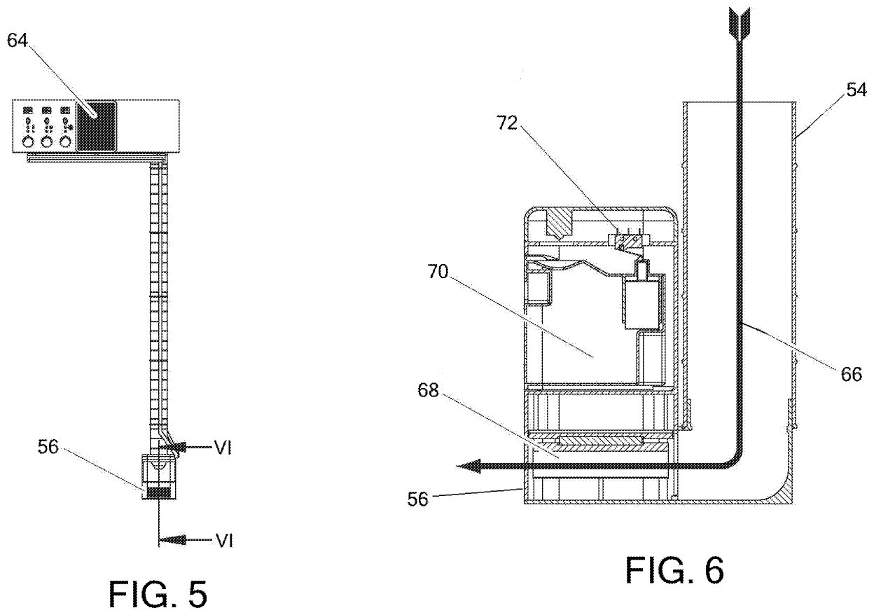

Referring now to FIGS. 4 through 10, a modular unit 50 for retro-fitting into an existing closet is shown. It should be understood that features discussed with reference to FIGS. 4 through 10 may be applicable to any other of the embodiments described herein. For example, the switch 48 may be included with the modular unit 50, even though it is not specifically shown in the Figures.

The modular unit 50 may fit into a closet to create a space having a function and design similar to closet 10 described above. The modular unit 50 can include a dehumidifier unit 52 connected via a duct 54 to an air discharge grill 56. A condensate drain line 58 may travel adjacent the duct 54 and may carry condensate from the dehumidifier unit 52 to a condensate collection unit 60. Similar to that described above, a sanitizer unit 62 may be disposed as part of the modular unit 50.

Air may travel along an air flow path 66 through the air intake grill 64, as shown in FIG. 6. Also shown is a heat transfer element 68 disposed adjacent the air discharge grill 56. Of course, the heat transfer element 68 may be located adjacent the dehumidifier unit 52, as is the case with closet 10 described above. Moreover, closet 10, described above, may position the heat transfer element 68 near the air discharge grill.

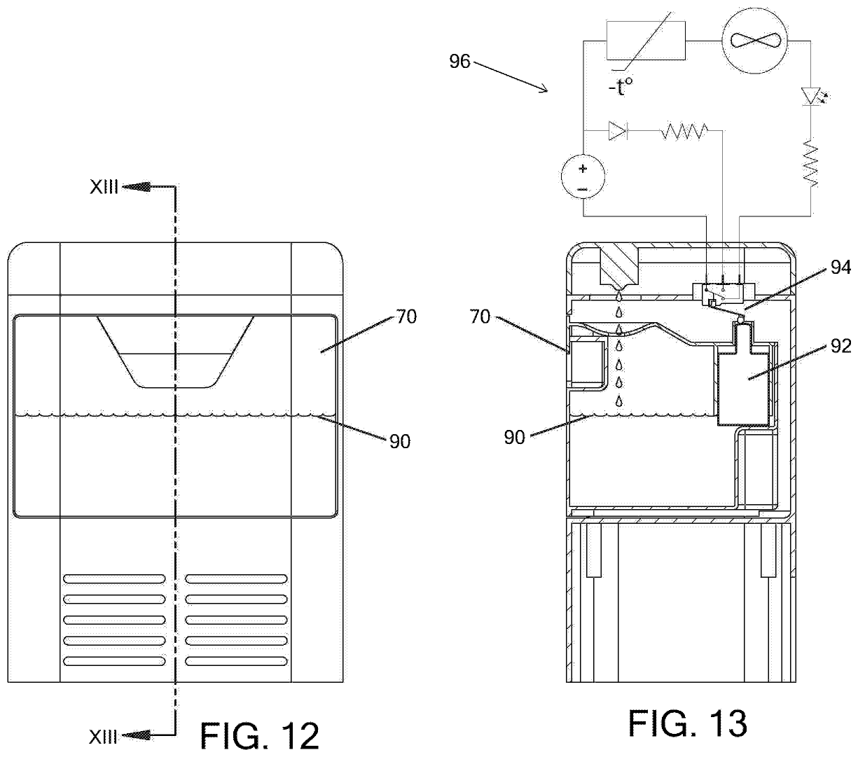

FIG. 6A further shows a detail view of a condensate container 70 having a switch valve 72 that may operate to turn off the system when the condensate container 70 is full. Details of this system is described below with respect to FIGS. 12 through 15.

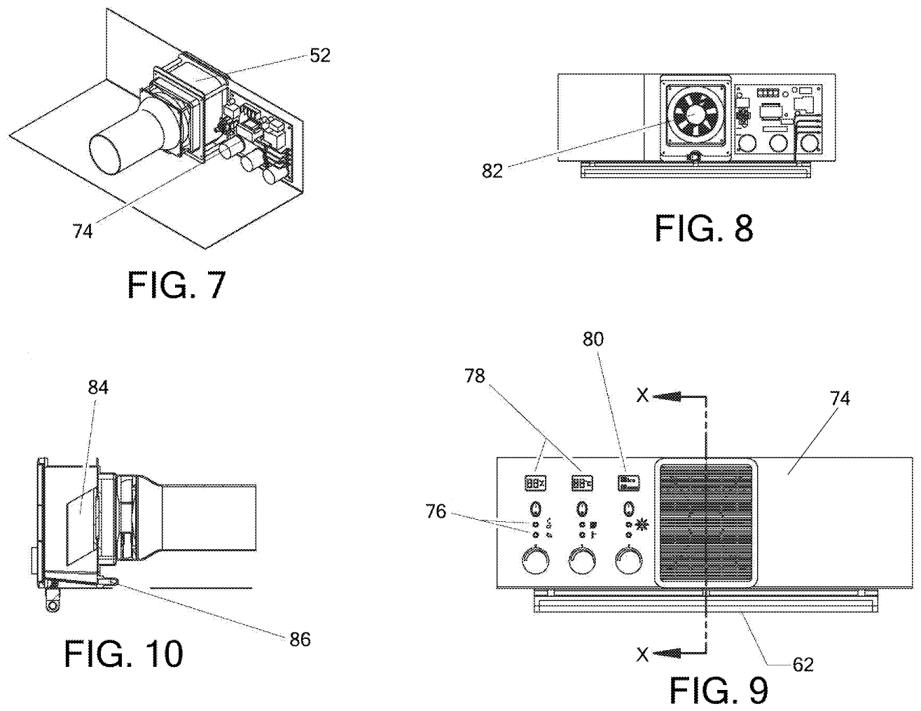

As shown in FIGS. 7 through 9, a control module 74 may include various features to control the system. The description of the control module 74 may also apply to the functions of the control module 30 of the closet 10 described above. The control module 74 may include indicators 76, such as LEDs, that may indicate that the system is operating, or that the condensate container 70 is full. Displays 78 may be used to show the relative humidity in the closet and heat settings. An additional display 80 may show the time the unit has run, or the time left to run, as well as a means to set a timer for operation thereof.

A circulating fan 82 may direct air to pass over the condensing elements 84 and into the duct 54. As shown, a condensate drain line connector 86 may be disposed below the condensing elements 84 so that the condensate drain line 58 can extend therefrom to the condensate container 70.

Referring now to FIGS. 12 through 15, a condensate fill operation is shown. These figures illustrate one embodiment of how control of the apparel drying system may be controlled to prevent condensate overflow. Moreover, it should be understood that, in some embodiments, the condensate container 70 may include a drain that is normally open to drain away condensate. In this embodiment, the condensate fill operation herein may be omitted, or may be used as a safety feature, should the normally open drain become blocked.

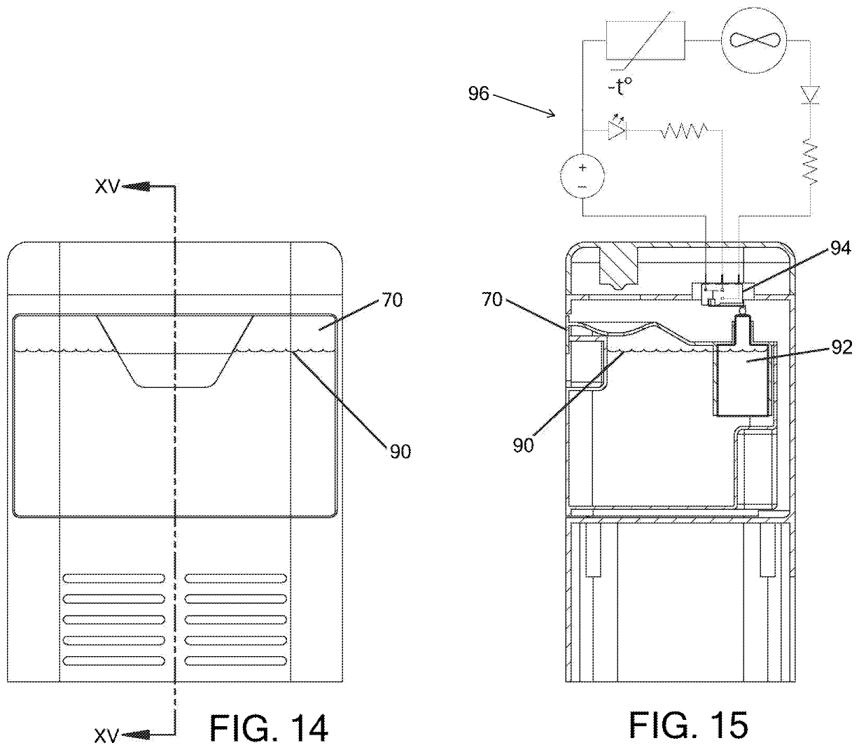

FIGS. 12 and 13 illustrate a condensate level 90 in the condensate container 70 below a filled level. In this embodiment, a float member 92 is not floating and a switch 94 enables operation of the system, as shown in the circuit 96. FIGS. 14 and 15 illustrate the condensate level 90 in the condensate container 70 in a filled level. In this embodiment, the float member 92 floats in the condensate 98 to activate the switch 94 to operate the circuit 96 in a matter that shuts down the apparel drying system.

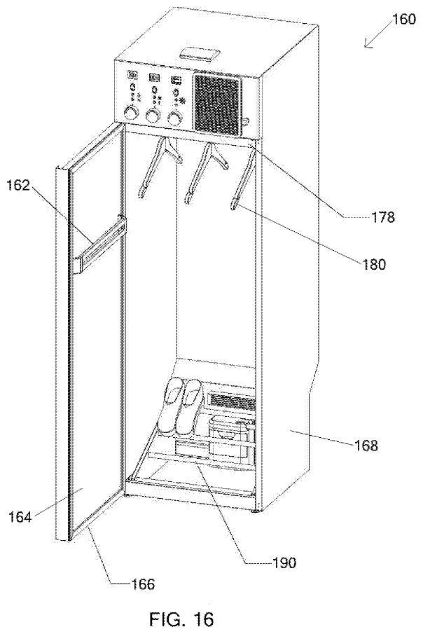

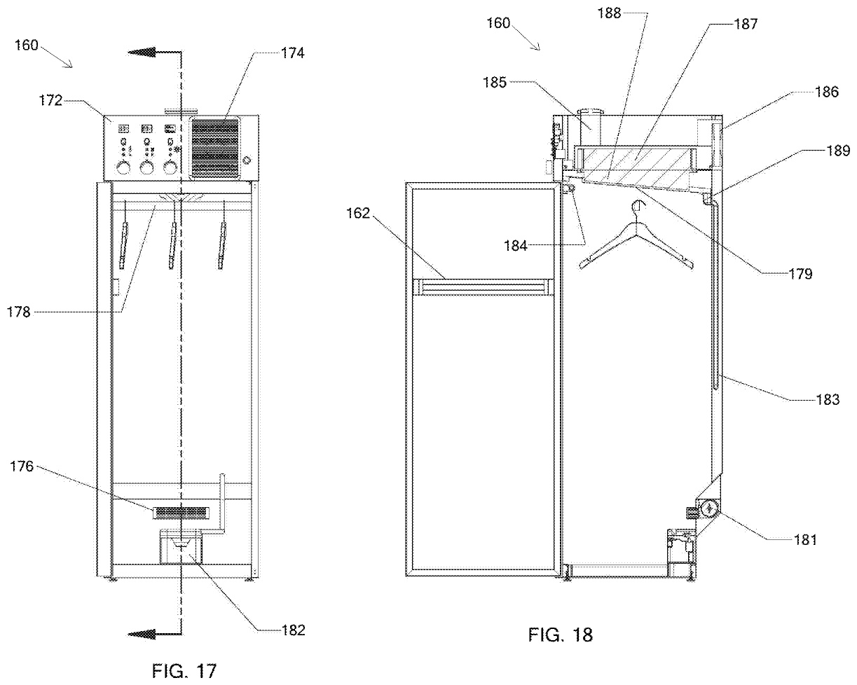

FIGS. 16 through 18 illustrate an apparel cabinet 160, also referred to a closet 160, that may include the features described above with respect to closet 10. It should be understood that the features described herein for closet 160 may be used in closet 10, and the features of closet 10 may be used in closet 160. The closet 160 can include a towel bar 162 mounted on the inside of the door 164 for hanging one or more towels thereon. The door 164 can include a door seal 166, such as a magnetic door seal, for providing a seal between the door 164 and the closet body 168. A shoe rack insert 170 may be disposed near a bottom of the closet 160 for placement of shoes. The shoe rack insert 170 may be removable as desired by the user. A hanger rod 178 may support hangers 180 thereon.

The closet 160 includes a control panel 172, similar to that described above with respect to closet 10. As discussed below, the air intake 174 may be used to bring external air into the closet 160. The discharge grate 176 may provide an output for dehumidified, heated and/or circulated air into the closet 160. A condensate collection vessel 182 may be provided to collect condensate, as described above with respect to closet 10, when the dehumidification feature is operational. A UV sanitizer light 184 may provide optional sanitization of air as it passes within the closet 160.

The closet 160 can include a cooling air intake 185 for intake of cooling air, a cooling core exhaust fan 186 for discharge of cooling air, a heat core evaporator 187 for providing cooling for dehumidification, and a cooling core vapor condenser 188 for condensation of water vapor in the dehumidification process. A drain pan 179 can guide condensate toward a drain cup 189 which passes the condensate to a drain line 183. A heater/blower 181 may be disposed at various locations in the closet 160. In the embodiment of FIG. 18, the heater/blower 181 is disposed adjacent the discharge grate 176.

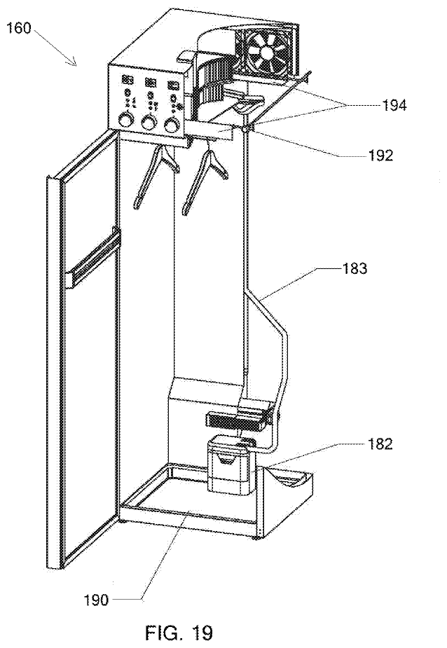

Referring to FIGS. 19 through 22, the closet 160 can include a drip pan 190 for collecting water that may drip from clothes or other items placed in the closet 160. The closet 160 can include one or more actuators 192 that can control cabinet air doors 194. The actuators 192 can open or close the cabinet air doors 194 to optionally permit external air to enter the closet 160 and for air to exit the closet 160. The actuator 192 and cabinet air doors 194 may be applied to any of the embodiments as herein described.

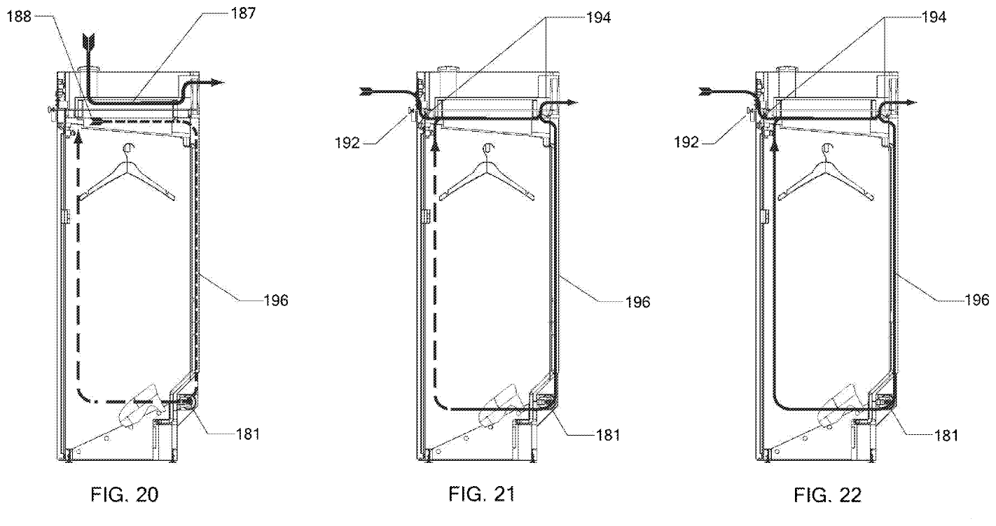

FIG. 20 shows one mode of operation where the cabinet air doors 194 are closed where the heater/blower 181 pushes dehumidified and/or heated air into the closet 160. The air may move up through the clothes (not shown) and pass into the cooling core vapor condenser 188, where the air is dehumidified and pulled through the return air duct 196. The operation mode shown in FIG. 20 may be referred to as an internal heat and dehumidification mode.

FIGS. 21 and 22 show additional modes of operation where the cabinet air doors 194 are open, permitting external air to flow in (as shown by arrow 198) and out (as shown by arrow 199) of the closet 160. In the operation mode of FIG. 21, the heater/blower 181 may provide heated air circulation. In the operation mode of FIG. 22, the heater/blower 181 may simply circulate air without heating.

In FIGS. 20 through 22, the solid line shows flow of external air, while the long dashed line shows flow of heated air and the short dashed line shows flow of dehumidified air.

As shown above, the closet 160 (and the closet 10, described above) may be operated in various modes of operation, with and without external air exchange, with and without heating the air, and with and without dehumidification of the air.

Power may be provided for the apparel drying unit according to various known techniques. Where the system is deployed in a stand-alone closet, the power may be provided via a power cord that may be connected to a standard power receptacle, for example. A similar power design may be employed for a retro-fitted system. For built-in systems, and for some embodiments of stand-along or retro-fitted applications, power may be hardwired to the closet. For example, the top of the closet may be provided with a power conduit for extending a power cable (not shown) therein.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

Claim elements and steps herein may have been numbered and/or lettered solely as an aid in readability and understanding. Any such numbering and lettering in itself is not intended to and should not be taken to indicate the ordering of elements and/or steps in the claims.

Many alterations and modifications may be made by those having ordinary skill in the art without departing from the spirit and scope of the invention. Therefore, it must be understood that the illustrated embodiments have been set forth only for the purposes of examples and that they should not be taken as limiting the invention as defined by the following claims. For example, notwithstanding the fact that the elements of a claim are set forth below in a certain combination, it must be expressly understood that the invention includes other combinations of fewer, more or different ones of the disclosed elements.

The words used in this specification to describe the invention and its various embodiments are to be understood not only in the sense of their commonly defined meanings, but to include by special definition in this specification the generic structure, material or acts of which they represent a single species.

The definitions of the words or elements of the following claims are, therefore, defined in this specification to not only include the combination of elements which are literally set forth. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a sub combination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptually equivalent, what can be obviously substituted and also what incorporates the essential idea of the invention.

What is claimed is:

1. An apparel drying system comprising:
an interior space configured for receiving apparel therein;
a dehumidifying unit having at least one condensing element;
an air intake positioned at a first location in the interior space;
a fan moving air from the interior space, through the air intake and over the at least one condensing element; and
an air discharge grill receiving dehumidified air after passing through the dehumidifying unit, the air discharge grill positioned at a second location, within the interior space.

2. The apparel drying system of claim 1, further comprising a sanitizing unit emitting an ultraviolet light within the interior space.

3. The apparel drying system of claim 2, wherein the interior space includes at least one access door and a safety switch that turns off the sanitizing unit when the at least one access door is open.

4. The apparel drying system of claim 1, further comprising a condensate collection unit collecting condensate from the at least one condensing element.

5. The apparel drying system of claim 4, further comprising a condensate detection switch turning off at least the fan and the dehumidifying unit when a condensate container is at a filled level.

6. The apparel drying system of claim 1, further comprising a heat transfer element positioned downstream the dehumidifying unit, the heat transfer element conditioning a temperature of the air before being emitted out of the air discharge grill.

7. The apparel drying system of claim 1, wherein the interior space is part of a stand-alone closet.

8. The apparel drying system of claim 1, wherein the interior space is part of a closet into which the apparel drying system is built-in.

9. The apparel drying system of claim 1, wherein the interior space is an existing closet into which the apparel drying system is retro-fitted.

10. The apparel drying system of claim 9, further comprising a duct delivering airflow from the dehumidifying unit to the air discharge grill.

11. The apparel drying system of claim 1, wherein the air intake and the air discharge grill are disposed adjacent opposite ends of the interior space.

12. An apparel drying closet comprising:
an interior space configured for receiving apparel therein;
one or more doors for accessing the interior space;
a dehumidifying unit having at least one condensing element;
an air intake positioned at a first location in the interior space;
a fan moving air from the interior space, through the air intake and over the at least one condensing element;
an air discharge grill receiving dehumidified air after passing through the dehumidifying unit, the air discharge grill positioned at a second location, within the interior space;
a condensate collection unit collecting condensate from the at least one condensing element; and
a sanitizing unit emitting an ultraviolet light within the interior space.

13. The apparel drying closet of claim 12, further comprising a safety switch turning off the sanitizing unit when the at least one access door is open.

14. The apparel drying closet of claim 12, further comprising a heat transfer element positioned downstream the dehumidifying unit, the heat transfer element conditioning a temperature of the air before being emitted out of the air discharge grill.

15. The apparel drying closet of claim 12, wherein the air intake and the air discharge grill are disposed adjacent opposite ends of the interior space.

16. An apparel drying system comprising:
an interior space configured for receiving apparel therein;
an air intake positioned at a first location in the interior space;
an air discharge grill positioned at a second location within the interior space;
a fan moving air from the interior space, through the air intake and out of the air discharge grill; and
cabinet air doors openable to permit external air to exchange with air inside the interior space, the cabinet air doors closable to exclude external air from inside the interior space.

17. The apparel drying system of claim 16, further comprising a dehumidifying unit having at least one condensing element, the air passing through the dehumidifying unit before being delivered to the air discharge grill.

18. The apparel drying system of claim 16, further comprising a condensate collection unit collecting condensate from the at least one condensing element.

19. The apparel drying system of claim 16, further comprising a heat transfer element positioned upstream the air discharge grill, the heat transfer element conditioning a temperature of the air before being emitted out of the air discharge grill.

20. The apparel drying system of claim 16, wherein the air intake and the air discharge grill are disposed adjacent opposite ends of the interior space.
